(19) United States
(12) Patent Application Publication
(10) Pub. No.: US 2015/0080236 A1
(43) Pub. Date: Mar. 19, 2015

(54) METHOD OF SUB-CLASSIFYING BREAST CANCER TUMORS

(71) Applicant: KUWAIT UNIVERSITY, SAFAT (KW)

(72) Inventors: FAHD AL-MULLA, AL-YARMOUK (KW); JEAN PAUL THIERY, SINGAPORE (SG)

(73) Assignee: KUWAIT UNIVERSITY, SAFAT (KW)

(21) Appl. No.: 14/028,457

(22) Filed: Sep. 16, 2013

(51) Int. Cl. G01N 33/68

(52) U.S. Cl. CPC G01N 33/6893
USPC 506/7; 435/6.12; 435/7.1

(57) ABSTRACT

The method of sub-classifying breast cancer tumors profiles the expression of the Raf kinase inhibitor protein (RKIP) from a tissue sample from a cancerous primary breast tumor. The RKIP expression is profiled using its mRNA or by immunohistochemical protein quantifying methods in order to detect the level of RKIP in the breast cancer tissue sample. Based upon the RKIP expression profile, a sub-classification of cancer type may then be assigned. An RKIP expression of approximately 10.31 indicates basal carcinoma, an RKIP expression of approximately 10.04 indicates Claudin-low carcinoma, an RKIP expression of approximately 10.47 indicates Luminal-A carcinoma, an RKIP expression of approximately 10.44 indicates Luminal-B carcinoma, an RKIP expression of approximately 10.25 indicates HER2+ carcinoma, an RKIP expression of approximately 10.43 indicates ER+ carcinoma, and an RKIP expression of approximately 10.30 indicates ER− carcinoma.

### BACKGROUND OF THE INVENTION

1. Field of the Invention

The present invention relates to the classification of tumors, and particularly to a method of sub-classifying breast cancer tumors based upon Raf kinase inhibitor protein (RKIP) expression in a breast cancer tumor tissue sample.

2. Description of the Related Art

Breast cancer classification divides breast cancer into categories according to different schemes, each based on different criteria and serving a different purpose. The major categories are the histolopathological type, the grade of the tumor, the stage of the tumor, and the expression of proteins and genes. As knowledge of cancer cell biology develops, these classifications are updated.

The receptor status of breast cancers has traditionally been identified by immunohistochemistry (IHC), which stains the cells based on the presence of estrogen receptors (ER), progesterone receptors (PR) and human epidermal growth factor receptor 2 (HER2). At present, this remains the most common method of testing for receptor status, but DNA multi-gene expression profiles can categorize breast cancers into molecular subtypes that generally correspond to IHC receptor status.

Receptor status is a critical assessment for all breast cancers, as it determines the suitability of using targeted treatments, such as tamoxifen and or trastuzumab. These treatments are now some of the most effective adjuvant treatments of breast cancer. Estrogen receptor positive (ER+) cancer cells depend on estrogen for their growth, so they can be treated with drugs to reduce either the effect of estrogen (e.g., tamoxifen) or the actual level of estrogen (e.g., aromatase inhibitors), and generally have a better prognosis. Generally, prior to modern treatments, HER+ had a worse prognosis, however HER2+ cancer cells respond to drugs, such as the monoclonal antibody trastuzumab (in combination with conventional chemotherapy), and this has improved the prognosis significantly. Conversely, triple negative cancer (i.e., no positive receptors) lacking targeted treatments now has a comparatively poor prognosis.

Androgen receptor is expressed in 80-90% of ER+ breast cancers and 40% of “triple negative” breast cancers. Activation of androgen receptors appears to suppress breast cancer growth in ER+ cancer, while in the ER− breast, it appears to act as a growth promoter. Efforts are presently underway to utilize this as prognostic marker and treatment

Receptor status was traditionally considered by reviewing each individual receptor (ER, PR, HER2) in turn, but newer approaches look at these together, along with the tumor grade, to categorize breast cancer into several conceptual molecular classes that have different prognoses and may have different responses to specific therapies. DNA microarrays have assisted this approach. Proposed molecular subtypes include: Basal-like (ER−, PR− and HER2−, also called triple negative breast cancer (TNBC); most BRCA1 breast cancers are basal-like TNBC); Luminal A (ER+ and low grade); Luminal B (ER+ but often high grade); (Luminal ER−/AR+; overlapping with apocrine and so-called “molecular apocrine”, a recently identified androgen responsive subtype that may respond to anti-hormonal treatment with bicalutamide); ERBB2/HER2+ (has amplified HER2/neu); Normal breast-like; and Claudin-low (a more recently described class, often triple-negative, but distinct in that there is low expression of cell-cell junction proteins, including E-cadherin and frequently there is infiltration with lymphocytes).

Thus, a method of sub-classifying breast cancer tumors and providing a prognosis based on RKIP expression solving the aforementioned problems is desired.

### SUMMARY OF THE INVENTION

The method of sub-classifying breast cancer tumors profiles the expression of the Raf kinase inhibitor protein (RKIP) from a tissue sample from a cancerous primary breast tumor. The RKIP expression is profiled using its mRNA (using reverse transcription polymerase chain reaction (RTPCR), microarrays or any other suitable type of expression test) or by immunohistochemical protein quantifying methods in order to detect the level of RKIP in the breast cancer tissue sample. Based upon the RKIP expression profile, a sub-classification of cancer type may then be assigned. An RKIP expression of approximately 10.31 indicates basal carcinoma, an RKIP expression of approximately 10.04 indicates Claudin-low carcinoma, an RKIP expression of approximately 10.47 indicates Luminal-A carcinoma, an RKIP expression of approximately 10.44 indicates Luminal-B carcinoma, an RKIP expression of approximately 10.25 indicates HER2+ carcinoma, an RKIP expression of approximately 10.43 indicates ER+ carcinoma, and an RKIP expression of approximately 10.30 indicates ER− carcinoma.

A reduced RKIP expression profile identifies patients at risk for cancer relapse, and vice versa, particularly in the Luminal A sub-classification. A relatively high RKIP expression profile may be used to identify Luminal A type breast cancer with a relatively good prognosis. However, a relatively low RKIP expression is found to be associated with Claudin-low, HER2-enriched, basal, Luminal B and estrogen negative sub-classes (with a poor prognosis). Thus, overall, RKIP expression may be used as an aid in the molecular sub-classification of breast cancer, and an increased RKIP expression profile may be used to identify patients with a good prognostic signature, regardless of sub-class. Further, it should be noted that increasing RKIP expression levels in breast cancer tissues may aid in breast cancer treatment.

These and other features of the present invention will become readily apparent upon further review of the following specification and drawings.

### BRIEF DESCRIPTION OF THE DRAWINGS

FIG. 1A is a plot comparing Raf kinase inhibitor protein (RKIP) expression profiles for basal, Claudin-low and Luminal cancerous breast tumor tissue samples.

FIG. 1B is a plot comparing RKIP expression profiles for basal, Claudin-low, Luminal A, Luminal B, ERBB2 and normal breast-like tissue samples.

FIG. 1C is a plot comparing epithelial-mesenchymal transition (EMT) signature score against RKIP gene expression in sample cell lines.

FIG. 1D is a plot comparing epithelial-mesenchymal transition (EMT) signature score against RKIP gene expression in clinical breast cancer tissue samples.

FIG. 2A is a Kaplan-Meier survival curve plotting patient survival against cancer-free survival years for Luminal breast cancer, comparing high RKIP expression against low RKIP expression.

FIG. 2B is a Kaplan-Meier survival curve plotting patient survival against cancer-free survival years for Luminal A breast cancer, comparing high RKIP expression against low RKIP expression.

FIG. 2C is a Kaplan-Meier survival curve plotting patient survival against cancer-free survival years for Luminal B breast cancer, comparing high RKIP expression against low RKIP expression.

FIG. 2D is a Kaplan-Meier survival curve plotting patient survival against cancer-free survival years for ER+ breast cancer, comparing high RKIP expression against low RKIP expression.

FIG. 2E is a Kaplan-Meier survival curve plotting patient survival against cancer-free survival years for ER+ breast cancer, comparing RKIP expression in the highest quartile against RKIP expression in the lowest quartile.

FIG. 2F is a Kaplan-Meier survival curve plotting patient survival against cancer-free survival years for ER− breast cancer, comparing high RKIP expression against low RKIP expression.

FIG. 2G is a Kaplan-Meier survival curve plotting patient survival against cancer-free survival years for ER− breast cancer, comparing RKIP expression in the highest quartile against RKIP expression in the lowest quartile.

Similar reference characters denote corresponding features consistently throughout the attached drawings.

### DETAILED DESCRIPTION OF THE PREFERRED EMBODIMENTS

The method of sub-classifying breast cancer tumors profiles the expression of the Raf kinase inhibitor protein (RKIP) from a tissue sample from a cancerous primary breast tumor. The RKIP expression is profiled using its mRNA (using reverse transcription polymerase chain reaction (RTPCR), microarrays or any other suitable type of expression test) or by immunohistochemical protein quantifying methods in order to detect the level of RKIP in the breast cancer tissue sample.

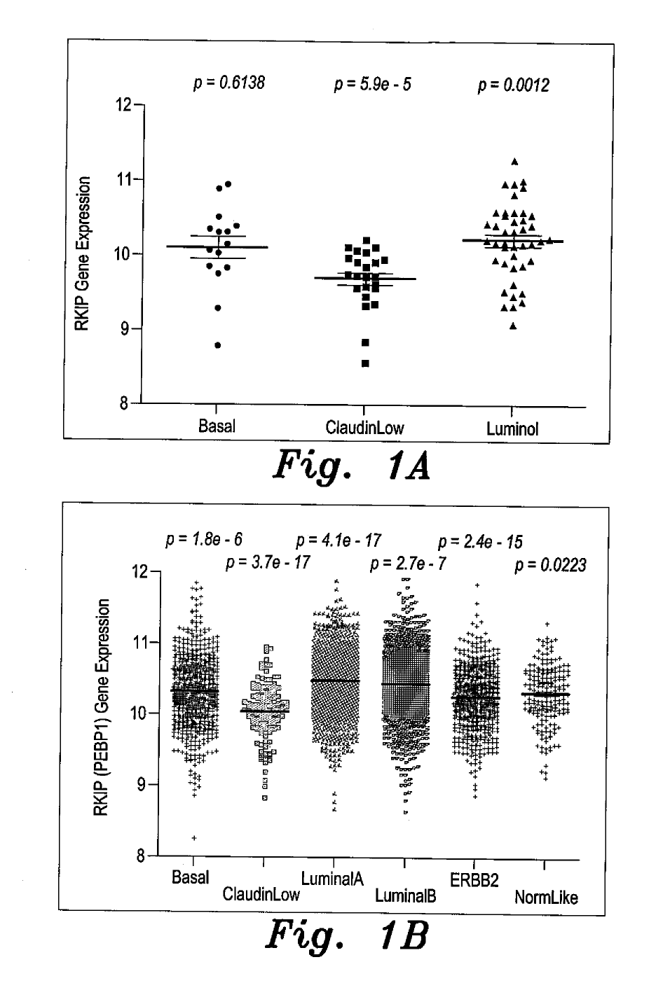

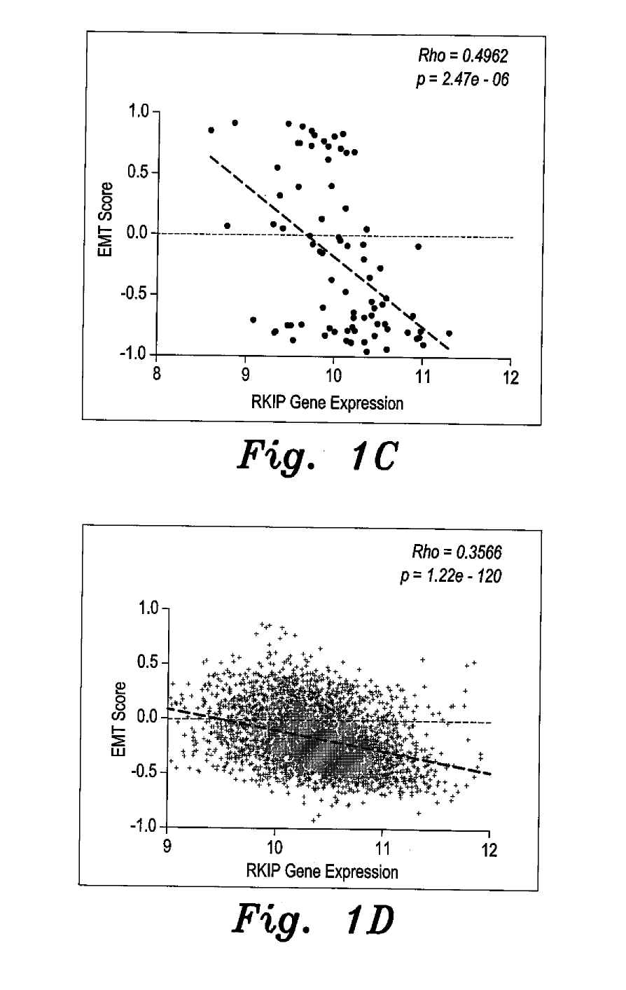

FIG. 1A illustrates a broad classification scheme based on RKIP expression. As shown in FIG. 1A, and as given below in Table 1, the mean RKIP (measured here in normalized RKIP expression units) can be used to categorize breast cancer tissue as basal, Claudin-low or Luminal carcinomas, based on 81 breast cancer cell line samples.

TABLE 1
Sub-Classification of Breast Cancer Based
on RKIP Expression in Cancer Cell Lines

| Sub-classification | Mean (95% CI) | Standard Error in Mean (SEM) |
|---|---|---|
| Basal | 10.1 (9.79-10.41) | 0.144 |
| Claudin-low | 9.7 (9.53-9.87) | 0.083 |
| Luminal | 10.22 (10.06-10.37) | 0.078 |

FIG. 1B and Table 2, below, show a more detailed sub-classification scheme using RKIP expression profiles for sub-classification into basal, Claudin-low, Luminal A, Luminal B, ERBB2 and normal breast-like carcinomas, based on molecular sub-classification from clinical breast cancer cases.

TABLE 2
Sub-Classification of Breast Cancer Based on RKIP
Expression From Clinical Breast Cancer Cases

| Sub-classification | Mean (95% CI) | Standard Error in Mean (SEM) |
|---|---|---|
| Basal | 10.31 (10.27-10.35) | 0.02 |
| Claudin-low | 10.04 (9.97-10.11) | 0.034 |
| Luminal-A | 10.47 (10.45-10.49) | 0.011 |
| Luminal-B | 10.44 (10.41-10.47) | 0.014 |
| HER2+ | 10.25 (10.21-10.28) | 0.019 |
| Normal breast- | 10.31 (10.26-10.37) | 0.029 |
| like | | |

Table 3 below shows further results for an additional sampling to determine ER+ and ER− breast carcinomas.

TABLE 3
Sub-Classification of ER+ and ER−
Breast Cancer Based on RKIP Expression

| Sub-classification | Mean (95% CI) | Standard Error in Mean (SEM) |
|---|---|---|
| ER+ | 10.43 (10.41-10.45) | 0.01 |
| ER− | 10.30 (10.27-10.33) | 0.015 |

FIGS. 1C and 1D illustrate the inverse correlation between RKIP expression and epithelial-mesenchymal transition (EMT) signature score against RKIP gene expression in the 81 breast cancer cell lines of FIG. 1A and Table 1, and in the clinical breast cancer cases of FIG. 1B and Table 2, respectively. In FIGS. 1A-1D, the statistical p values compare each sub-class with the rest of the sub-classes. In FIGS. 1C and 1D, rho statistics were used to establish the linear correlation between RKIP expression and EMT.

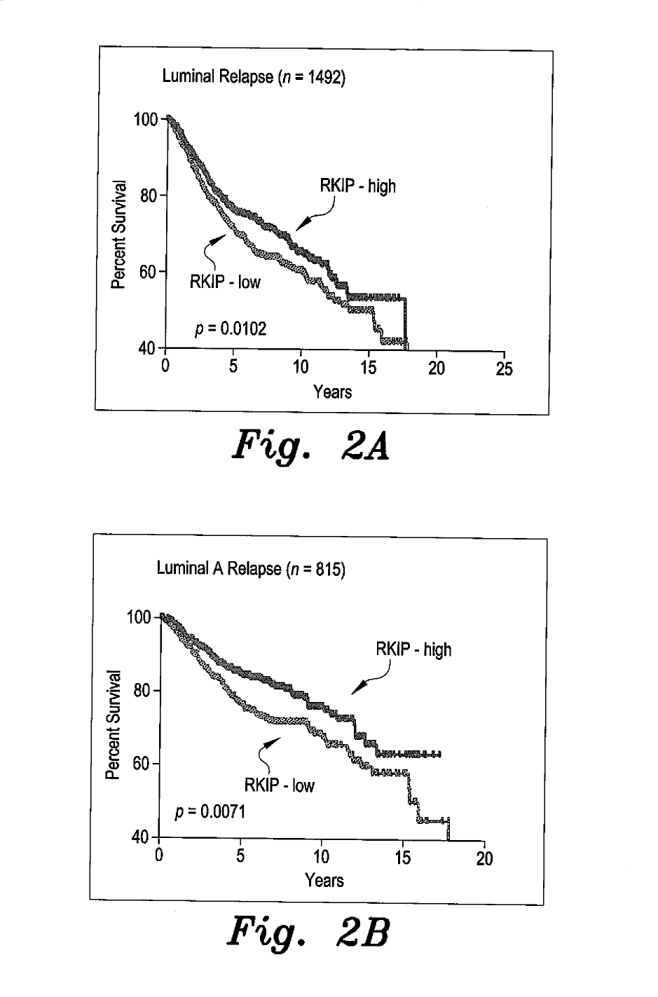

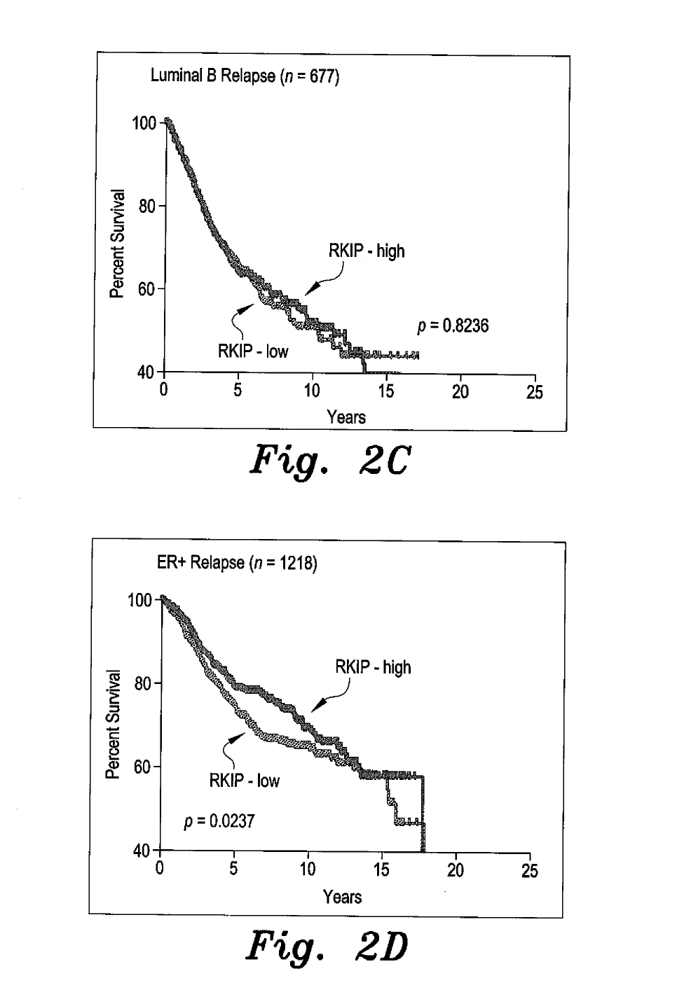

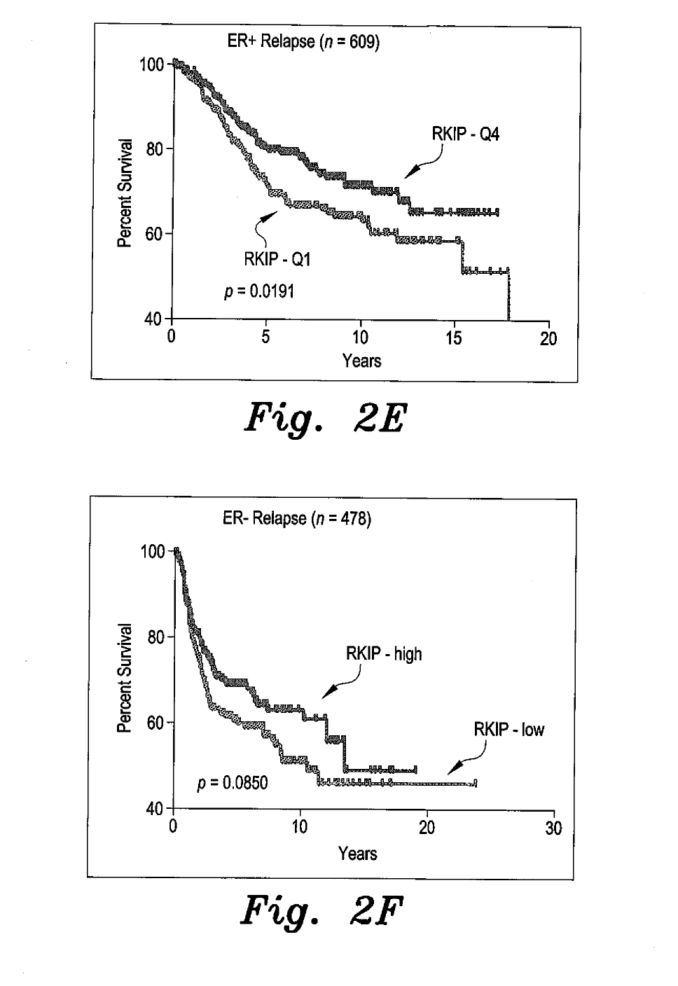

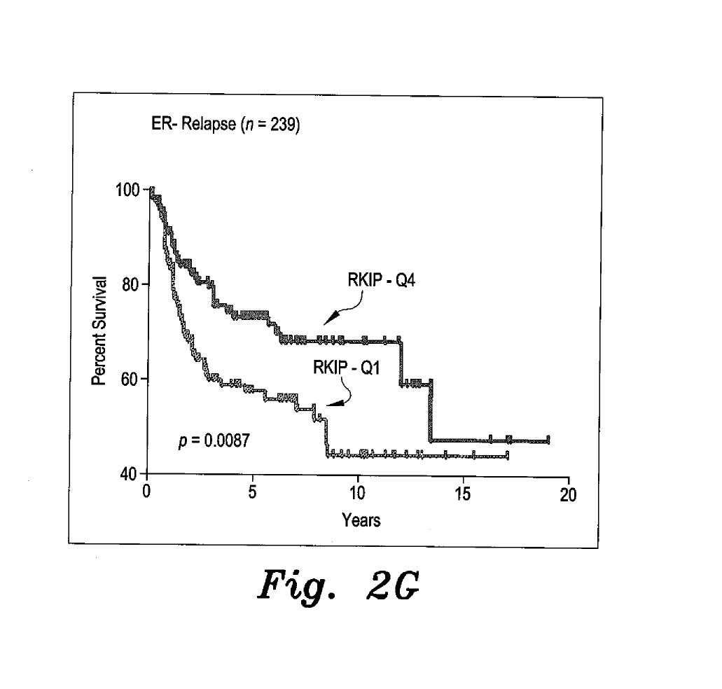

In addition to using RKIP expression values to sub-classify breast cancer types, the RKIP expression may be further used to generate a prognosis for a particular patient. FIGS. 2A-2G present Kaplan-Meier survival curves plotting patient survival against cancer-free survival years (based on mean time for relapse). As shown in each case, a relatively high RKIP expression profile provides a relatively good prognosis for a patient (i.e., low chance or relapse), with the number of survival years being extended when compared against a relatively low RKIP expression profile (offering a poor prognosis with an increased chance of relapse). FIG. 2A shows the comparison for Luminal breast cancer for a sample of 1,492 patients. FIG. 2B shows a similar comparison for Luminal A breast cancer, with a sample population of 815 patients. FIG. 2C shows the comparison for Luminal B breast cancer, with a sample size of 677 patients. FIG. 2D illustrates the comparison for ER+ breast cancer, with a sample size of 1,218 patients. FIG. 2E also illustrates ER+ breast cancer, but further comparing RKIP expression in the highest quartile against RKIP expression in the lowest quartile for a sample size of 609. FIG. 2F illustrates the comparison for ER− breast cancer with a sample size of 478 patients. FIG. 2G also illustrates ER− breast cancer, but further comparing RKIP expression in the highest quartile against RKIP expression in the lowest quartile for a sample size of 239.

It should be noted that the above experiments further illustrated that estrogen appears to induce the expression of RKIP. As shown above, a reduced RKIP expression profile identifies patients at risk for cancer relapse and vice versa, particularly in the Luminal A sub-classification. A relatively high RKIP expression profile may be used to identify Luminal A type breast cancer with a relatively good prognosis. However, a relatively low RKIP expression is found to be associated with Claudin-low, HER2-enriched, basal, Luminal B and estrogen negative sub-classes (with a poor prognosis). Thus, overall, RKIP expression may be used as an aid in the molecular sub-classification of breast cancer, and an increased RKIP expression profile may be used to identify patients with a good prognostic signature, regardless of sub-class. Further, it should be noted that increasing RKIP expression levels in breast cancer tissues may aid in breast cancer treatment.

It is to be understood that the present invention is not limited to the embodiments described above, but encompasses any and all embodiments within the scope of the following claims.

We claim:

1. A method of sub-classifying breast cancer tumors based on RKIP expression, comprising the steps of:
taking a tissue sample from a breast cancer tumor;
profiling Raf kinase inhibitor protein (RKIP) expression in the tissue sample; and
assigning a sub-classification of cancer type based on the RKIP expression profile, wherein an RKIP expression of approximately 10.31 indicates basal carcinoma, an RKIP expression of approximately 10.04 indicates Claudin-low carcinoma, an RKIP expression of approximately 10.47 indicates Luminal-A carcinoma, an RKIP expression of approximately 10.44 indicates Luminal-B carcinoma, an RKIP expression of approximately 10.25 indicates HER2+ carcinoma, an RKIP expression of approximately 10.43 indicates ER+ carcinoma, and an RKIP expression of approximately 10.30 indicates ER− carcinoma.

2. The method of sub-classifying breast cancer tumors based on RKIP expression as recited in claim 1, wherein the step of profiling the RKIP expression in the tissue sample comprises reverse transcription polymerase chain reaction mRNA analysis.

3. The method of sub-classifying breast cancer tumors based on RKIP expression as recited in claim 1, wherein the step of profiling the RKIP expression in the tissue sample comprises microarray mRNA analysis.

4. The method of sub-classifying breast cancer tumors based on RKIP expression as recited in claim 1, wherein the step of profiling the RKIP expression in the tissue sample comprises immunohistochemical protein quantification.

5. The method of sub-classifying breast cancer tumors based on RKIP expression as recited in claim 1, further comprising the step of generating a patient prognosis based upon the RKIP expression profile.
